(19) United States
(12) Patent Application Publication
(10) Pub. No.: US 2015/0119946 A1
(43) Pub. Date: Apr. 30, 2015

(54) Community Orthopedic Fixation Device, System and Method

(71) Applicant: Jace Medical, LLC, Winona Lake, IN (US)

(72) Inventors: Justin James May, Leesburg, IN (US); Scott Steffensmeier, Winona Lake, IN (US)

(73) Assignee: Jace Medical, LLC, Winona Lake, IN (US)

(21) Appl. No.: 14/524,705

(22) Filed: Oct. 27, 2014

Related U.S. Application Data
(60) Provisional application No. 61/896,318, filed on Oct. 28, 2013.

(51) Int. Cl. A61B 17/84

(52) U.S. Cl. CPC A61B 17/842; A61B 17/846; A61B 2017/681
USPC 606/328

(57) ABSTRACT

An orthopaedic fixation system for closing a resection of a body part, including a plurality of anchors located on either side of a resection which separates a body part into adjacent portions, such that there are a set of first anchors on one side of the resection and a second set of anchors on the other side of the resection. A first cable member is wound around and between the first and second sets of anchors, with the cable member extending generally orthogonally across the resection between at least certain pairs of the first and second anchors, with one of the pairs of anchors comprising a node.

### CROSS REFERENCES TO RELATED APPLICATIONS

This application claims the benefit of U.S. Provisional Patent Application Ser. No. 61/896,318, filed Oct. 28, 2013, the content of which is incorporated herein by reference in its entirety.

### FIELD

The disclosure relates generally to orthopaedic fixation apparatus, systems and methods of such fixation, and particularly regarding use in bone fixation.

### BACKGROUND

In some surgical procedures involving bones, for instance, the procedure may involve separating a bone into portions, which are thereafter reunited. This happens, for instance, in entries into the chest cavity, as for heart surgery, where the sternum is required to be separated along its length, in the transverse plane, or a combination of the two. There may be other instances where a bone has undergone fracturing through some trauma, and is thereafter to have portions rejoined for proper healing.

The bones or skeletal tissue, or combinations of bone and tissue, can be held secure to one another in adjacency using a fixation device, or system. The system is desirably designed that in the event that subsequent surgery is required, as in an emergency reentry to the chest cavity, the fixation device may be opened quickly and easily with as little harm to the patient as possible. Many kinds of conventional fixation devices include wires or cables that are organized to pull the bone portions together, laterally across a divide or fracture. These types of fixation devices can be relatively complex to emplace, and require more effort than desired to undue in the event of an emergency.

### SUMMARY

An orthopaedic fixation system for closing a resection of a body part is disclosed. The system may include a plurality of anchors located on either side of a resection which separates a body part into adjacent portions, such that there are a set of first anchors on one side of the resection and a second set of anchors on the other side of the resection, with first and second anchors generally located in opposed relationship across the resection. A first cable member is wound around and between the first and second sets of anchors, with the cable member extending generally orthogonally across the resection between at least certain pairs of the first and second anchors, with one of the pairs of anchors comprising a node.

A second cable member may be wound around and between the first and second sets of anchors in a pattern different from a pattern of winding used with the first cable. A plurality of bands may be included, wherein each band surrounds a perimeter defined by a group of first and second anchors, the bands being spaced apart along the resection.

Another orthopaedic fixation system for closing a resection of a body part is disclosed. The system may include a plurality of anchors located on either side of a resection which separates a body part into adjacent portions, such that there are a set of first anchors on one side of the resection and a second set of anchors on the other side of the resection. A first cable member may be wound around and between the first and second sets of anchors in a first winding arrangement, and a second cable member may be wound around and between the first and second sets of anchors in a winding arrangement different from the winding arrangement used with the first cable.

Yet another orthopaedic fixation system for closing a resection of a body part is disclosed. This system includes a plurality of anchors located on either side of a resection which separates a body part into adjacent portions, such that there are a set of first anchors on one side of the resection and a second set of anchors on the other side of the resection. A first cable member may be wound around and between the first and second sets of anchors in a first winding arrangement. A plurality of bands may also be included, wherein each the band surrounds a perimeter defined by a group of first and second anchors, the bands being spaced apart along the resection.

### BRIEF DESCRIPTION OF THE DRAWINGS

Embodiments of apparatus, systems, and methods are illustrated in the figures of the accompanying drawings which are meant to be exemplary and not limiting, in which like references are intended to refer to like or corresponding parts, and in which:

FIG. 1 is a front plan view of an orthopaedic fixation system made in accordance with an aspect of the disclosure;

FIG. 2 is a front plan view of an orthopaedic fixation system made in accordance with another aspect of the disclosure; and

FIG. 3 is a front plan view of an orthopaedic fixation system made in accordance with yet another aspect of the disclosure.

### DETAILED DESCRIPTION

While the embodiments described hereinafter are in the environment of an orthopaedic fixation apparatus, system and method for use on the sternum, it should be appreciated that the disclosure has broader application, such as where bone or other body parts having suitable rigidity require closure or other relational organization, such as joining two opposing anatomical structures. Thus, the present disclosure can have usefulness in contexts beyond fixation of bones.

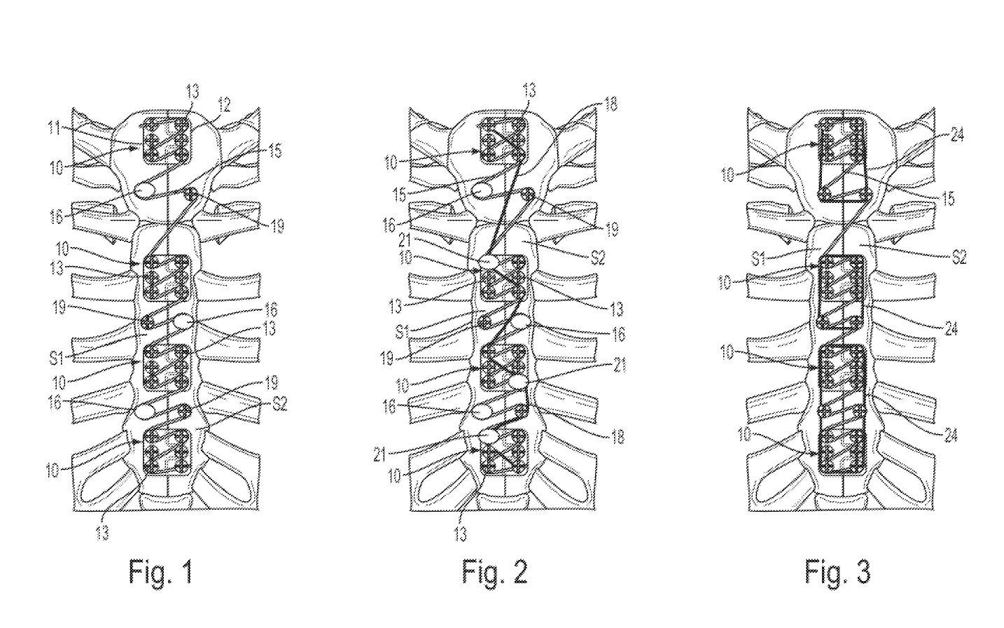

FIG. 1 illustrates a distributed tensioning system using what will be referred to as a “community” concept for the tether or tying arrangement. “Community” expresses the use of different anchoring concepts in a system. What is illustrated is a plurality of interlocking plates or retainers 10. The retainers 10 span the sternum portions S1 and S2. In a surgical procedure, the sternum portions S1 and S2 are separated, and then must be rejoined.

A first portion 11 of a retainer 10 is coupled on one sternum portion, such as sternum portion S1, and a second portion 12 of the retainer 10 is coupled on an opposed part of the other sternum portion, such as sternum portion S2. The first and second portions 11 and 12 may be interlocking or interengaging parts that are fixed into place with fasteners, such as bone screws 13, known in the art. As should be understood, however, many of the modules, plates and other fixation devices described and contemplated herein may be anchored using other anchor elements besides screws, such as staples, rivets, nails or other fasteners of the like. Screws are generally described herein, as typically the more desirable anchor in a particular embodiment.

As illustrated in FIG. 1, four retainers 10 are employed, each having a first portion 11 coupled on one sternum portion, such as sternum portion S1, and a second portion 12 of the retainer 10 is coupled on an opposed part of the other sternum portion, such as sternum portion S2. Each of the first and second portions include at least one anchor, such as a screw 13. Thus, a plurality of anchors 13 are located on either side of the resection which separates the sternum or other body part into adjacent portions.

A first set of first anchors 13 are on one side of the resection, such as sternum portion S1, and a second set of anchors 13 on the other side of the resection, such as sternum portion S2. Respective pairs of first and second anchors 13 are generally located in opposed relationship across the resection.

A single cable or tether 15 is used to engage the first and second portions 11 and 12 (i.e., anchors) of the various retainers 10, and also link the retainers 10 along the sternum. The tether 15 may be woven through, around, or otherwise engage the screws 13. The terms tether and cable are used interchangeably herein. For example, as illustrated, a first tether 15 is wound around and between the first and second sets of anchors 13, with the tether 15 extending generally orthogonally across the resection between at least certain pairs of the first and second anchors 13.

This embodiment may also use particular node elements 16 that act as anchors between respective retainers 10 along the sternum. The tether 15 is locked at these node elements 16. This makes the system less dependent on the integrity of each node. The system may also make use of isolated screw mounts 19, which also act as anchors, between respective retainers 10 through which, or around which, the tether 15 is woven in a desired arrangement or first winding arrangement.

In another embodiment similar to that of FIG. 1, a double tether arrangement is employed in FIG. 2. This is similar to what is shown in FIG. 1, with the first tether 15 being locked at the three nodes 16. A second tether 18 is used, which is wound through or around various ones of the screws 13 (i.e. anchors), and fixed at additional node points 21. For example, as illustrated, the second tether 18 may be wound around and between the first and second sets of anchors 13, 16, and 19 in a pattern different from a pattern of winding used with the first tether 15. This system is less dependent on the integrity of each node and the tethers.

In yet another embodiment of the FIG. 1 type of community concept, FIG. 3 illustrates a tether arrangement like that of FIG. 1, without the node fixation (although it could easily also include the same). Here, however, bands or perimeter tethers 24 are disposed around selected areas including a retainer plate 10.

For example, as illustrated in FIG. 3, a plurality of perimeter tethers 24 may be included, wherein each perimeter tether 24 surrounds a perimeter defined by a group of first and second anchors. The perimeter tethers 24 are spaced apart along the resection. These isolated perimeter tethers 24 provide high load sharing potential, and once again provide a system which is less dependent on the integrity of the each node or each retainer 10.

Although the apparatus, systems, and methods have been described and illustrated in connection with certain embodiments, many variations and modifications should be evident to those skilled in the art and may be made without departing from the spirit and scope of the disclosure. The discourse is thus not to be limited to the precise details of methodology or construction set forth above as such variations and modification are intended to be included within the scope of the disclosure.

What is claimed is:

1. An orthopaedic fixation system for closing a resection of a body part, comprising:
a plurality of anchors located on either side of a resection which separates a body part into adjacent portions, such that there are a set of first anchors on one side of the resection and a second set of anchors on the other side of the resection, with first and second anchors generally located in opposed relationship across the resection; and
a first cable member wound around and between the first and second sets of anchors, with the cable member extending generally orthogonally across the resection between at least certain pairs of the first and second anchors, with one of the pairs of anchors comprising a node.

2. The fixation system of claim 1, further comprising a second cable member which is wound around and between the first and second sets of anchors in a pattern different from a pattern of winding used with the first cable.

3. The fixation system of claim 1, further comprising a plurality of bands, each the band surrounding a perimeter defined by a group of first and second anchors, the bands being spaced apart along the resection.

4. An orthopaedic fixation system for closing a resection of a body part, comprising:
a plurality of anchors located on either side of a resection which separates a body part into adjacent portions, such that there are a set of first anchors on one side of the resection and a second set of anchors on the other side of the resection;
a first cable member wound around and between the first and second sets of anchors in a first winding arrangement; and
a second cable member is wound around and between the first and second sets of anchors in a winding arrangement different from the winding arrangement used with the first cable.

5. An orthopaedic fixation system for closing a resection of a body part, comprising:
a plurality of anchors located on either side of a resection which separates a body part into adjacent portions, such that there are a set of first anchors on one side of the resection and a second set of anchors on the other side of the resection;
a first cable member wound around and between the first and second sets of anchors in a first winding arrangement; and
a plurality of bands, each the band surrounding a perimeter defined by a group of first and second anchors, the bands being spaced apart along the resection.
